(19) United States
(12) Patent Application Publication
(10) Pub. No.: US 2022/0117835 A1
(43) Pub. Date: Apr. 21, 2022

(54) Skin Stimulation Tool

(71) Applicant: Johnson Consulting, LLC, San Juan, PR (US)

(72) Inventor: Benjamin Johnson, Evergreen, CO (US)

(21) Appl. No.: 17/451,108

(22) Filed: Oct. 15, 2021

Related U.S. Application Data
(60) Provisional application No. 63/198,414, filed on Oct. 16, 2020.

(51) Int. Cl. A61H 15/02; A61M 37/00; A61H 15/00

(52) U.S. Cl. CPC A61H 15/02; A61M 37/00; A61H 15/0092; A61H 2201/1695; A61H 2015/0042; A61H 2201/105; A61M 2037/0007

(57) ABSTRACT

A skin stimulation tool to increase penetration and efficacy of topically applied products and medications. An example of the skin stimulation tool includes a substantially Y-shaped handle. The example skin stimulation tool also includes a first attachment on a first branch of the Y-shaped handle, and a second attachment on a second branch of the Y-shaped handle. The example skin stimulation tool also includes a first head portion rotatably mounted on the first attachment of the handle, and a second head portion rotatably mounted on the second attachment of the handle. Both of the head portions rotate when applied to a surface of the skin under linear motion of the handle.

### PRIORITY CLAIM

This application claims the priority filing benefit of U.S. Provisional Patent Application No. 63/198,414 filed Oct. 16, 2020 of Benjamin Johnson for “Skin Stimulation Tool,” hereby incorporated by reference for all that is disclosed as though fully set forth herein.

### BACKGROUND

Various skin serums, powders, lotions, and other treatments are widely available to treat a number of skin conditions and for skin enhancement. While the skin may absorb a portion of these products when applied topically, larger quantities may be required to deliver an effective dose. The size of the molecules can determine how well an ingredient penetrates. In addition, some users may receive a higher effective dose, while others receive a lower effective dose, due to the ability of their skin to absorb the treatment product(s).

The skin has a unique design that makes the penetration of ingredients challenging. The lipids interwoven on the surface of the epidermis tend to repel water and water-soluble ingredients. Research indicates that between 2-5% of topically applied ingredients penetrate with even lower percentages for molecules larger than 500 Daltons. Most strategies that attempt to increase penetration rely on the destruction of a healthy, protective barrier, thereby increasing water loss, sun damage, and sensitizing the skin.

Devices and chemicals have been used to increase skin penetration. These include micro-needling, acids, micro-dermabrasion, scrubs, exfoliants, alcohol, and other techniques. But these all compromise the health of the skin and the protective capacity of the barrier each time they are employed. This either causes instant harm to the skin, or increases skin sensitivity with repeated efforts. Damaging the barrier increases environmental toxin exposure, dehydrates the skin, and can increase future UV damage from the sun because the barrier that serves as a partial reflector of the sun's rays is weakened.

### BRIEF DESCRIPTION OF THE DRAWINGS

FIGS. 1-2 are perspective views of an example skin stimulation tool.

FIG. 3 is a side perspective view of an example skin stimulation tool.

FIG. 4 is a bottom perspective view of an example skin stimulation tool.

FIG. 5 is a top perspective view of an example skin stimulation tool.

FIG. 6 is a top view of an example skin stimulation tool showing example dimensions.

FIG. 7 is a side view of an example skin stimulation tool showing example dimensions.

FIG. 8 is a perspective view of an example head of the skin stimulation tool.

FIG. 9 is a top view of the head shown in FIG. 8.

FIG. 10 is a side view of the head shown in FIG. 8.

### DETAILED DESCRIPTION

A skin stimulation tool is disclosed herein. In an example, the skin stimulation tool includes a handle and at least one head portion. The head portion rolls on the user's skin as the handle is moved in a linear motion. The head portion may include a plurality of pyramid or other shapes to raise the surface of the head. These raised surfaces when rolled across the skin opens the pores and causes the skin to become more receptive to a topical treatment. For example, the skin stimulation tool may be used before, during, and/or after applying one or more skin treatment(s), such as skin treatment serum(s) or other topical agent.

The skin stimulation tool is uniquely designed to enable passage or penetration of the topical agent, by creating temporary micro-channels or pathways in the skin. In an example, these pathways are created by a raised surface, such as small pyramidal shaped wedges that separate the corneocytes and lipids at the skin surface in such a way that enables the penetration of ingredients by increasing the allowable size of molecules of the topical agent (e.g., to approximately 1000 Daltons).

In an example, the raised surface (e.g., pyramidal wedges) work in conjunction with and take advantage of the flexibility of the lipid/corneocyte surface of the epidermis, rather than removing constituent parts of the skin like other penetration-enhancing strategies. That is, the raised surface separates the layers of stratum corneum, but only temporarily, thereby avoiding long term structural damage to the skin.

Before continuing, it is noted that as used herein, the terms “includes” and “including” mean, but is not limited to, “includes” or “including” and “includes at least” or “including at least.” The term “based on” means “based on” and “based at least in part on.”

It is also noted that the examples described herein are provided for purposes of illustration, and are not intended to be limiting. Other devices and/or device configurations may be utilized to carry out the operations described herein.

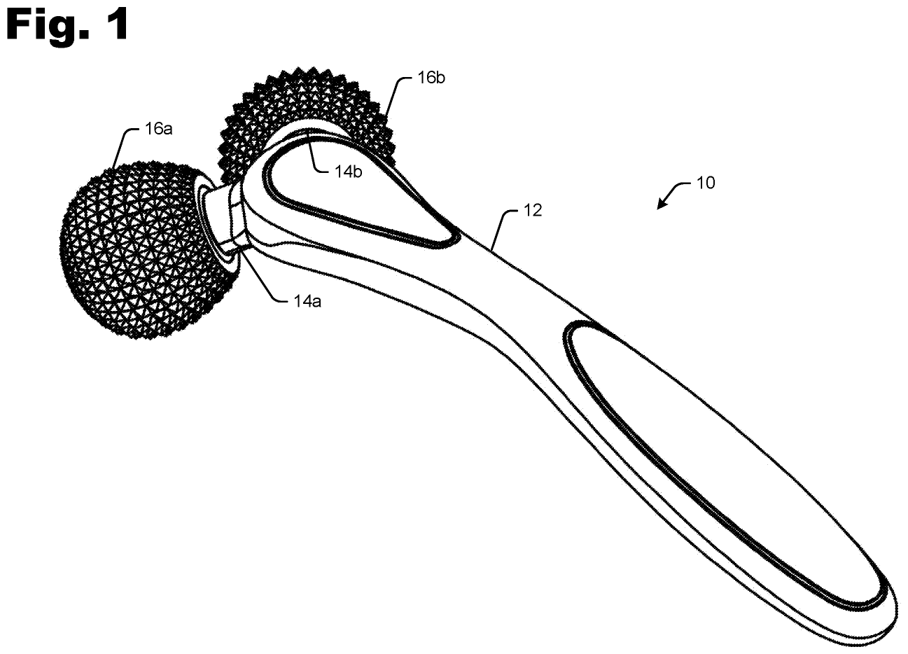

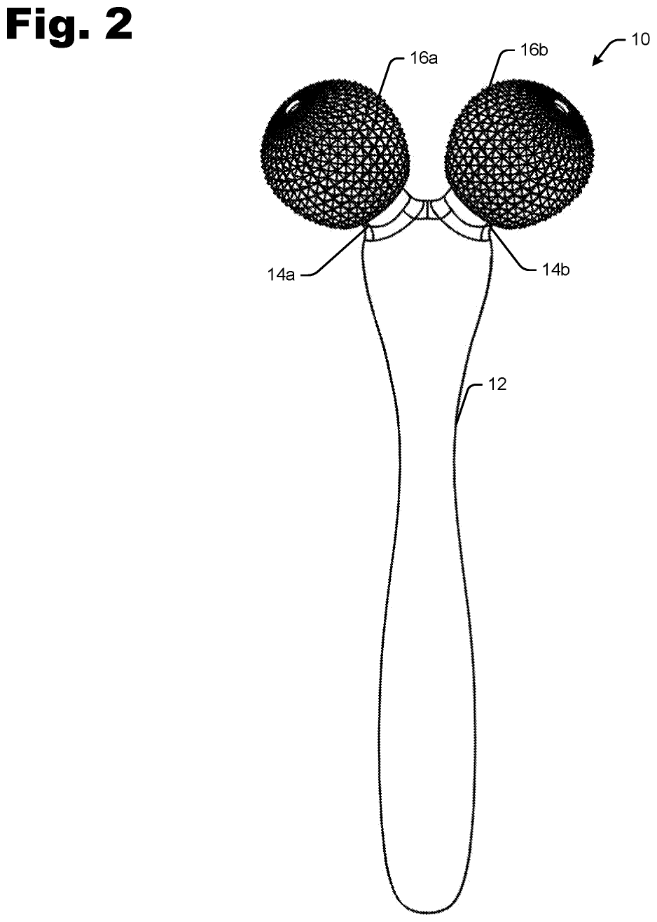

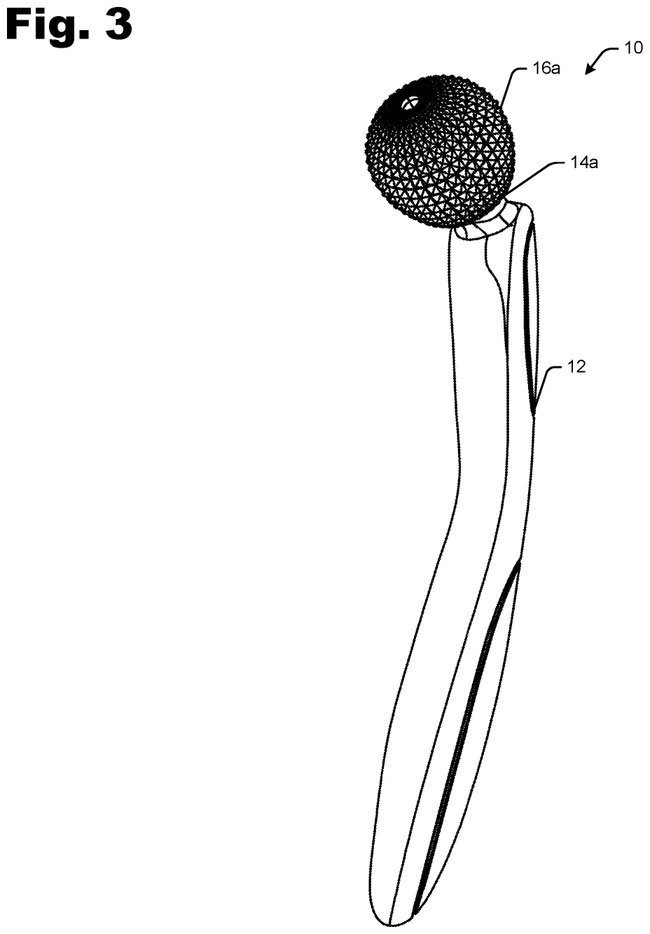

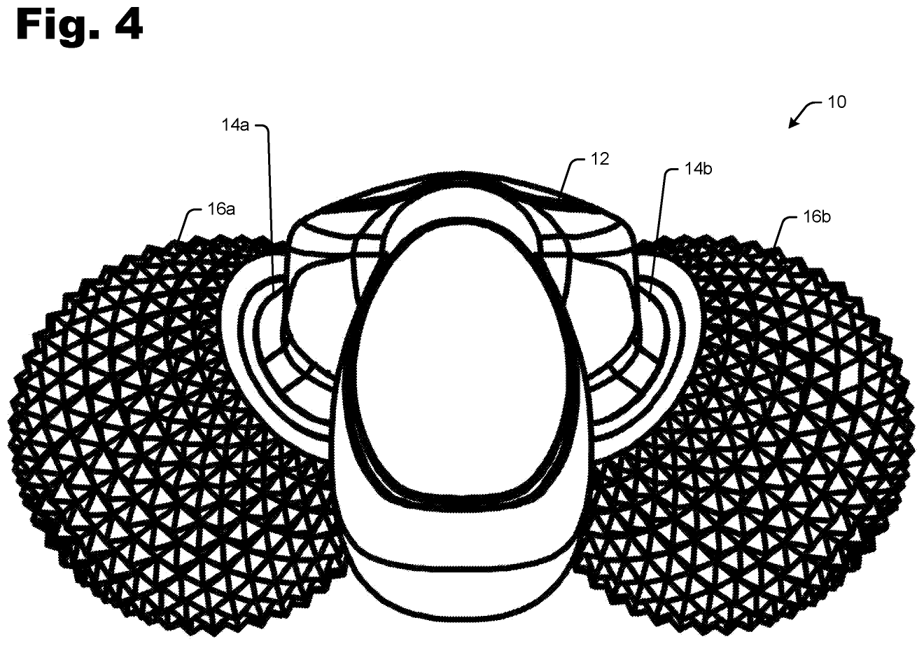

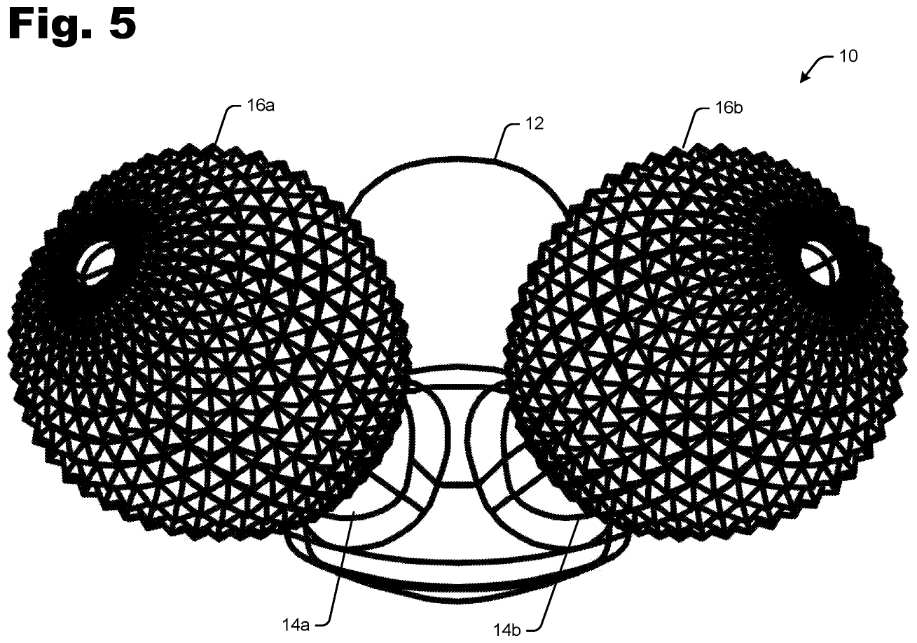

FIGS. 1-2 are perspective views of an example skin stimulation tool 10. FIG. 3 is a side perspective view of an example skin stimulation tool 10. FIG. 4 is a bottom perspective view of an example skin stimulation tool 10. FIG. 5 is a top perspective view of an example skin stimulation tool 10.

In an example, the skin stimulation tool 10 may be implemented to increase penetration and efficacy of topically applied products and medications. An example of the skin stimulation tool 10 includes a substantially Y-shaped handle 12, or a handle portion having one or more head connections, a first attachment 14a on a first branch of the Y-shaped handle 12, and a second attachment 14b on a second branch of the Y-shaped handle 12. The example skin stimulation tool 10 also includes a first head portion 16a rotatably mounted on the first attachment 14a of the handle 12, and a second head portion 16b rotatably mounted on the second attachment 14b of the handle 12. Both of the head portions 16a and 16b are configured to rotate when applied to a surface of the skin under a linear (forward and/or backward, e.g., back-and forth) motion of the handle 12.

In an example, the skin stimulation tool 10 is entirely manually operated. That is, the handle 12 may be held in the hand of a human operator and pushed and/or pulled, while applying at least some pressure with the head portions 16a and 16b in contact with the skin. This causes the head portions 16a and 16b to automatically rotate (clockwise and/or counterclockwise). In an example, this rotation is entirely in response to the head portions 16a and 16b rolling across the surface of the skin. In another example, rotation may be effected at least in part via a motor.

It will be understood by those having ordinary skill in the art after becoming familiar with the teachings herein that the tool is not limited to any particular number of head portions. In FIG. 1, two head portions are illustrated by way of example. But other examples may include a single head portion or more than two head portions, depending at least to some extent on design considerations and application.

In an example, the head portions 16a and 16b are substantially spherical in shape. However, the head portion need not be spherical. Other shapes (e.g., cylindrical, ovoid, cube, etc.) may also be suitable, again depending at least to some extent on design considerations and application.

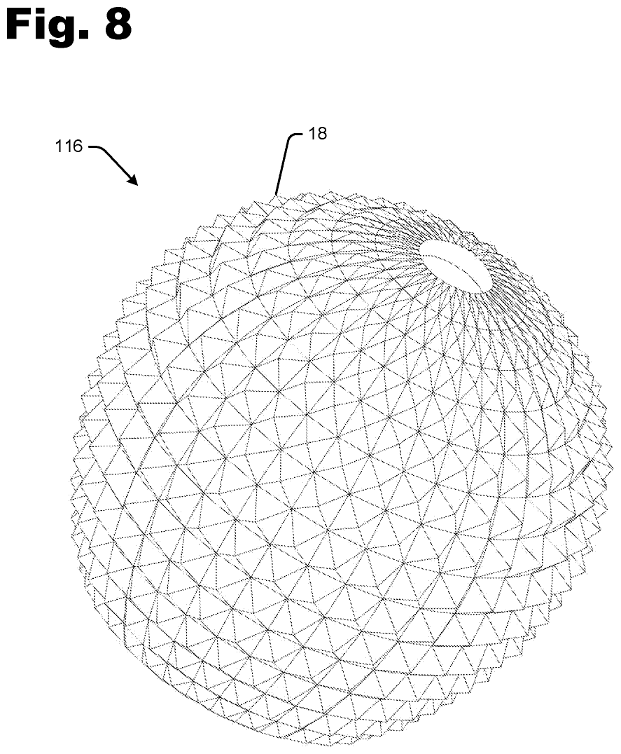

In an example, the head portions 16a and 16b include a raised surface 18. While any suitable raised surface may be provided, an example raised surface includes a plurality of pyramids (or “micro-pyramids”) substantially covering the surface of a sphere-shaped head portion. These pyramids will be discussed in more detail below with reference to the specific examples shown in FIGS. 8-10. In other examples, different shapes may be provided on the same head portion 16a, 16b and/or different shapes may be provided on different head portions 16a, 16b. For example, the head portions 16a, 16b may be interchangeable on the handle 12, enabling the end-user to swap out the head portions 16a, 16b on the same handle for different end-uses and/or skin types.

The skin stimulation tool 10 may be manufactured of any suitable material. In an example, the skin stimulation tool 10 is manufactured as a molded polymer (e.g., plastic), and has a coating applied thereto. The coating may be selected to further enhance operation on the skin. For example, the coating may be a metalized finish. In other examples, the skin stimulation tool 10 may be manufactured of steel or other durable material. Different components of the skin stimulation tool 10 may also be manufactured of different types of materials. In an example, the skin stimulation tool 10 is manufactured of material that is both cost effective and easy to manufacture, such as a polymer structure with a copper alloy electroplated surface coating.

In an example, the skin stimulation tool 10 is specifically designed for use twice daily without causing damage to the skin, to aid in use with common skincare applications that are intended to occur twice daily (e.g., once in the morning, and again at nighttime). Unlike traditional needles that may be used for a processing known as “needling” which disrupts the skin barrier, the skin stimulation tool 10 only has a pointed tip that may be the size of the needle. But the raised surface or pyramid progressively widens as toward the base of the pyramid. This structure enables the pyramid to penetrate the skin, making the skin stimulation tool 10 a much more effective tool for skin penetration without causing damage to the skin.

It is noted that the unique design of the roller ball combined with the Y-shaped handle allows for easy transitions around bony protrusions on the face, such as cheekbones and eye sockets. The skin stimulation tool 10 can even be used on the eyelids safely and effectively. It is easy to treat the skin around the mandible and nose. Indeed, the skin stimulation tool 10 can be effectively used on any part of the body without causing damage to the skin while still increasing penetration for a topical agent that is applied to the skin after using the skin stimulation tool 10.

It is also noted that the skin stimulation tool 10 may have other applications. Example applications may include, but are not limited to, increasing efficacy for treatment with topical medications, as a relaxation or massage therapy tool, a tool to increase blood and/or lymphatic flow near the skin's surface, and treatment of itchy skin.

During operation, the handle is moved (e.g., linearly, in circular or sem-circular motions) to cause the head portions 16a and 16b to at least partially rotate on the surface of the skin. Moving the head portion(s) 16a and 16b across the skin causes the pores to open, forming pathways and making the skin more receptive to treatment via topical agent(s). It is noted that the skin stimulation tool 10 may be used before, during, and/or after applying one or more skin treatment(s).

The operations shown and described herein are provided to illustrate example implementations. It is noted that the operations are not limited to the ordering shown. Still other operations may also be implemented.

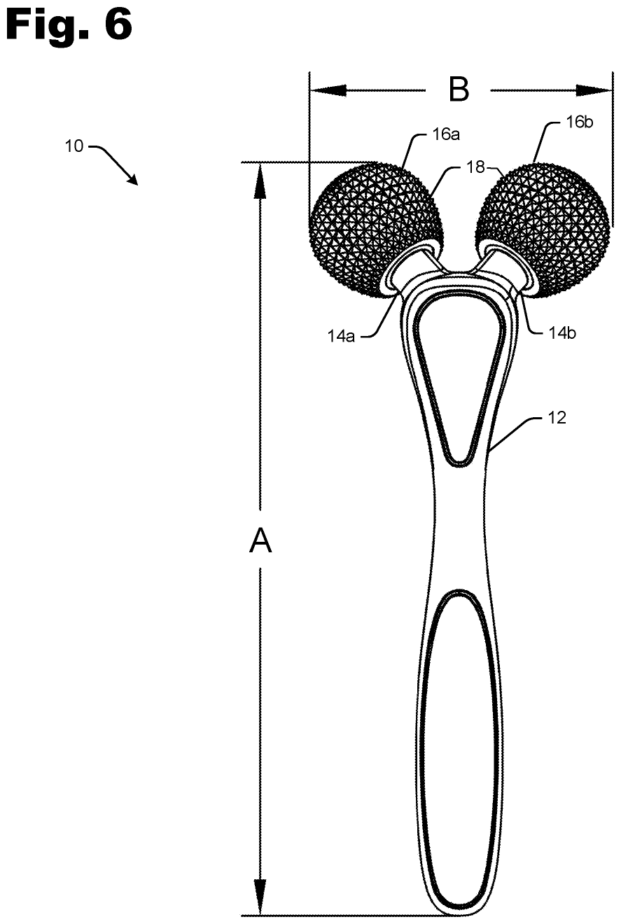

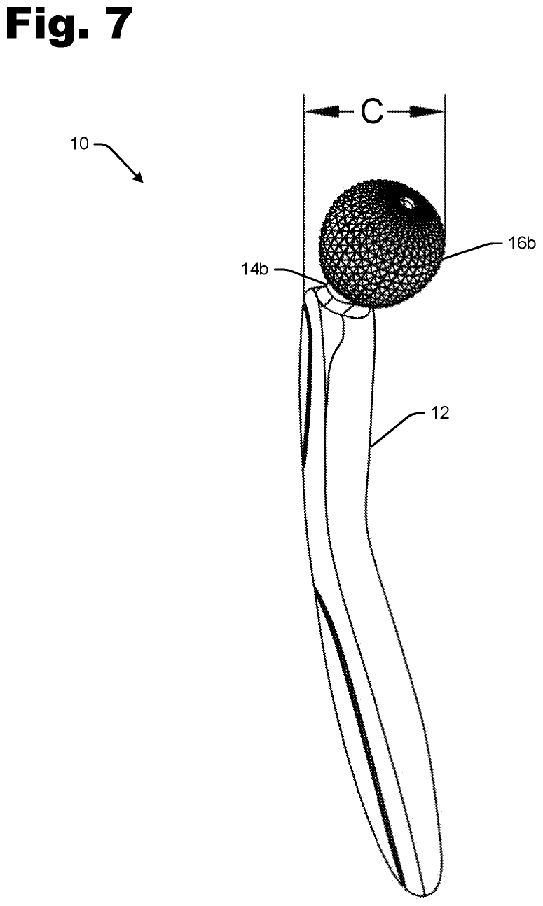

FIG. 6 is a top view of an example skin stimulation tool 10 showing example dimensions. In an example, the handle is about 13 cm in length. Each attachment arm for the head portions is about 3 cm in length. The dimension A shown in FIG. 6 is about 169 mm; and the dimension B shown in FIG. 6 is about 68 mm. FIG. 7 is a side view of an example skin stimulation tool 10 showing example dimensions. In an example, the dimension C shown in FIG. 7 is about 33 mm.

The dimensions noted above have been shown to create the an efficacious “grab” of the skin. That is, smaller dimensions do note accommodate the wedges as well, and larger dimensions make the tool 10 heavy and bulky, and the head portions 16a, 16b are too far apart to effectively “grab” at the skin. It is noted that the skin stimulation tool 10 is not limited to these dimensions, and the skin stimulation tool 10 may be manufactured with other dimensions that are greater than or less than those noted above.

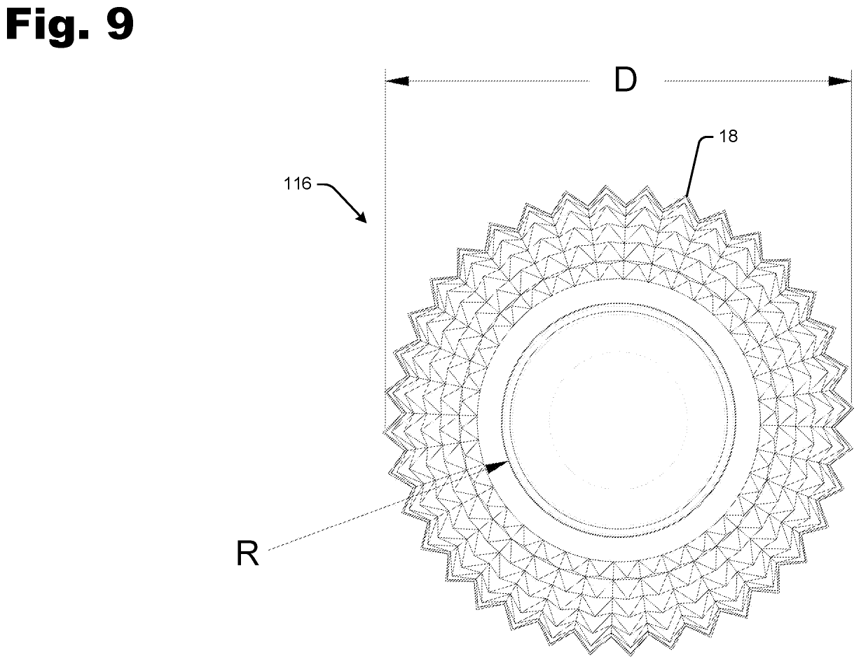

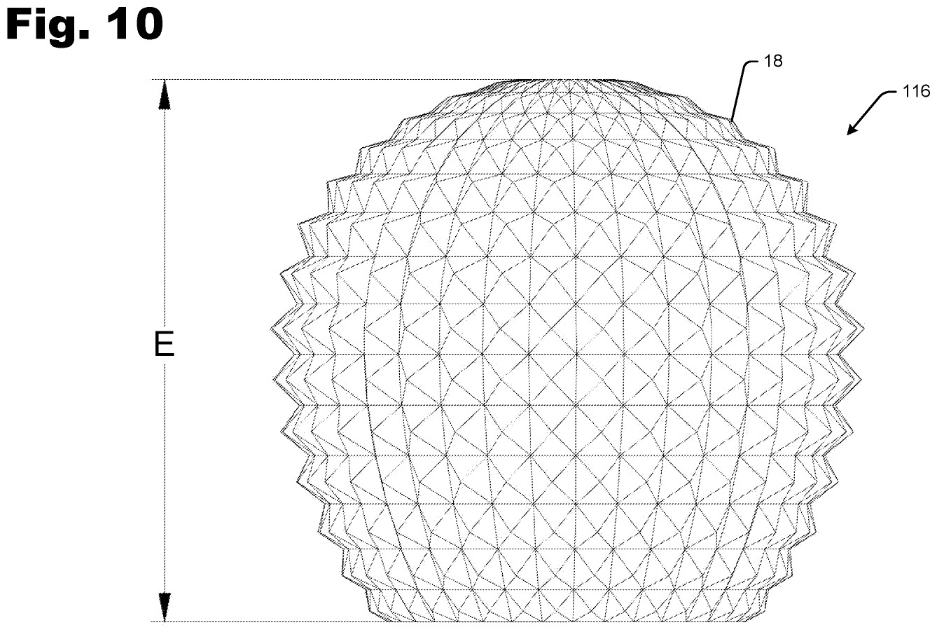

FIG. 8 is a perspective view of an example head 116 (e.g., heads 16a and 16b) of the skin stimulation tool 10. FIG. 9 is a top view of the head 116 shown in FIG. 8. FIG. 10 is a side view of the head 116 shown in FIG. 8. In an example, the preferred overall dimension of the ball is about 25 mm wide (dimension D shown in FIG. 9) and about 23 mm in height (dimension E shown in FIG. 10). However, a range in size may include about 10 mm to 25 mm wide and about 10 mm to 23 mm in height. The radius R (FIG. 9) of the opening in the bottom of the head 116 for fitting the head 116 onto the handle (e.g., attachments 14a, 14c of the handle 12) is about 6 mm. However, this dimension may also vary based on design considerations.

Example design considerations for the various dimensions include, but are not limited to, being able to provide the desired surface texture (e.g., the pyramids of the raised surface), the spread between the surface texture and the corresponding number of micro-channels that can be created with each pass of the tool 10. The example dimensions given herein were designed to maximize the number of micro-channels that can be effectively formed in the skin, without causing damage to the skin.

In an example, the skin stimulation tool 10 has a raised surface or surface texture. This texture may include many small and strategically positioned pyramid shaped wedges on a spherical surface. This design has been found to facilitate easy, complete coverage of the skin.

In an example, the pyramids or “wedges” range from about 0.5 mm to 0.7 mm in height. This height forms a micro-passage in the skin at a depth that only passes through the stratum corneum, but limits the impact on the more sensitive layers of skin beneath the stratum corneum (e.g., from top to bottom: stratum corneum, stratum granulosum, stratum spinosum, stratum basale).

In an example, each wedge is spaced approximately 2 millimeters apart from the adjacent wedge and forms a “progressive pattern”. This progressive pattern creates as many pathways as possible in the skin for maximal ingredient penetration. That is, the pyramids create a progressive widening (via the pointed tip, to the wider base) of the micro-channels formed in the skin, that further increases penetration of the topical agent. The wedges also serve to “grab” the skin (e.g., to pull upward on the skin as the heads are rolled across the surface of the skin). When used in combination with the “Y” shaped handle, this design has been shown to achieve the proper depth to enhance penetration of the topical agent without causing damage to the skin.

It is noted that ranges of depth or height of the wedges may be about 0.2 mm to about 1.2 mm. The depth or height of the wedges can vary based on the amount of pressure that is applied. Shallower wedges will require more pressure and deeper wedges may require less pressure to form the same size micro-channel in the skin. An example preferred range is about 0.5 mm to about 0.7 mm. This enables the user to make slight variations in pressure, while still providing the least amount of potential inflammation while enhancing penetration. These dimensions were found to create a slight pull or “grab” on the skin, especially in less bony areas of the face. The micro-channels reach just past the stratum corneum into the beginning of the stratum granulosum, bypassing the lipid layer for better penetration, while affecting as little of the stratum granulosum as possible.

It is noted that the examples shown and described are provided for purposes of illustration and are not intended to be limiting. Still other examples are also contemplated.

1. A skin stimulation tool to increase penetration and efficacy of topically applied products and medications, the skin stimulation tool comprising:
a substantially Y-shaped handle;
a first attachment on a first branch of the Y-shaped handle, and a second attachment on a second branch of the Y-shaped handle;
a first head portion mounted to rotate around the first attachment of the handle, and a second head portion independently mounted from the first head portion to rotate around the second attachment of the handle, both of the head portions rotating when applied to a surface of the skin under linear motion of the handle; and
a raised surface on each of the first and second head portions.

2. The skin stimulation tool of claim 1, wherein the handle has a length of about 13 cm, and each branch of the Y-shaped handle having a length of about 3 cm.

3. The skin stimulation tool of claim 1, wherein the head portions are each about 25 mm wide and about 23 mm tall.

4. The skin stimulation tool of claim 1, wherein the head portions are each about 10 mm to 25 mm wide and about 10 mm to 23 mm tall.

5. The skin stimulation tool of claim 1, wherein a distance between a top-most portion of each of the head portions and an end portion of the handle is about 169 mm.

6. The skin stimulation tool of claim 1, wherein a distance between an outermost surface of each of the first head portion and the second head portion is about 68 mm.

7. The skin stimulation tool of claim 1, wherein a distance between an inner most surface of each of the head portions and a back of the handle is about 33 mm.

8. The skin stimulation tool of claim 1, wherein the head portion is substantially spherical shaped.

9. The skin stimulation tool of claim 1, wherein the raised surface of the first and second head portions is formed as a plurality of distinct pyramids.

10. The skin stimulation tool of claim 9, wherein a top portion of each of the plurality of pyramids is a point, and sides of each of the plurality of pyramids progressively widen toward a base of the pyramids, wherein the shape of the pyramids is configured to open skin pores and form pathways into the skin as the pyramids are rolled over the skin, thereby increasing penetration of a topical agent as the head portions are pressed deeper into the skin.

11. The skin stimulation tool of claim 9, wherein the plurality of pyramids of the head portions are each sized to maximize formation of a number of micro-channels in contact with the skin during use.

12. The skin stimulation tool of claim 9, wherein a base of adjacent pyramids of the head portions are in contact with one another.

13. The skin stimulation tool of claim 9, wherein a base of adjacent pyramids of the head portions are spaced about 2 mm apart.

14. The skin stimulation tool of claim 9, wherein a height of each of the pyramids of the head portions is about 0.2 mm to 1.2 mm.

15. The skin stimulation tool of claim 9, wherein a height of each of the pyramids of the head portions is about 0.5 mm to 0.7 mm to create a depth that only passes through a stratum corneum of the skin, and limits impact on more sensitive skin layers.

16. The skin stimulation tool of claim 9, wherein each of the plurality of pyramids formed on a surface of both head portions is 4-sided.

17. The skin stimulation tool of claim 9, wherein each of the plurality of pyramids formed on a surface of both head portions is substantially the same size.

18. The skin stimulation tool of claim 9, wherein the plurality of pyramids formed on both head portions increases in size from a middle surface toward an upper surface and toward a lower surface of both head portions.

19. The skin stimulation tool of claim 1, wherein the raised surface works at a lipid-corneocyte surface of the skin to temporarily separate stratum corneum layers to create pathways into the skin without causing long term structural damage to the skin.

20. A skin stimulation tool to increase penetration and efficacy of topically applied products and medications, the skin stimulation tool comprising:
a substantially Y-shaped handle;
a first attachment on a first branch of the Y-shaped handle, and a second attachment on a second branch of the Y-shaped handle;
a first head portion rotatably mounted on the first attachment of the handle, and a second head portion rotatably mounted on the second attachment of the handle, both of the head portions rotating when applied to a surface of the skin under linear motion of the handle; and
a raised surface on each of the first and second head portions, the raised surface opening skin pores to form pathways into the skin, thereby increasing penetration of a topical agent as the head portions are pressed deeper into the skin without causing long term structural damage to the skin.
